(19) United States
(12) Patent Application Publication
(10) Pub. No.: US 2005/0152847 A1
(43) Pub. Date: Jul. 14, 2005

(54) Novel formulation

(76) Inventors: Eva Trofast, Lund (SE); Jan Trofast, Lund (SE)

Correspondence Address: FISH & RICHARDSON P.C., 225 FRANKLIN STREET, BOSTON, MA, 02110, US

(21) Appl. No.: 10/506,590

(22) PCT Filed: Mar. 3, 2003
(86) PCT No.: PCT/SE03/00371

(30) Foreign Application Priority Data
Mar. 4, 2002 (SE) 0200657-5

(51) Int. Cl. A61L 9/04; A61K 9/14; A61K 31/473; A61K 31/573

(52) U.S. Cl. 424/46; 514/171; 514/290

(57) ABSTRACT

The present invention relates to specific excipients for powder formulations for oral and nasal inhalation.

### FIELD OF THE INVENTION

The present invention relates to specific excipients for powder formulations for oral and nasal inhalation.

### BACKGROUND

The recent debate about transmissible spongiform encephalopathies (TSE) has highlighted the need for alternatives of excipients for use in pharmaceutical formulations. Compounds from an animal source should be abandoned in favour of compounds from the plant kingdom, or produced by effective and cheap synthetic procedures. Care has to be taken in the selection of new excipients since the drug delivery can be affected by the excipients through altered release of drug, bioavailability, solubility, stability, and dissolution rates leading to altered therapeutic activity and even an increase/decrease of unwanted side effects. Excipients are not always inert, and can show adverse toxicological findings by themselves or in drag formulations (see e.g. Br. J. Clin. Pharm (1988), 25, 283-287 and Resp. Med. (1990), 84, 345-348). An excipient should also fulfil all the physicochemical requirements as well as regulatory requirements necessary for a formulation in respiratory health care.

Sucrose is very moisture sensitive and will form cakes very easily when submitted to humidity and thereby being unsuitable as a constituent in formulations for inhalation. Its caries promoting effects make it also undesirable.

There are only two compounds presently on the market as carriers/diluents for inhalation formulations, namely lactose and glucose—both reducing saccharides. Besides, the main compound used is lactose which is isolated from the animal kingdom. A new excipient is therefore strongly needed.

WO95/00127 and WO95/00128 relate to polypeptide powders for inhalation, and disclose that non-reducing sugars such as raffimose, melezitose, lactitol, maltitol, trehalose, sucrose, mannitol and starch may be suitable additives for the polypeptide powders.

U.S. Pat. No. 6,004,574 describes a powder formulation for the administration of medically useful polypeptides, comprising a medically useful polypeptide with melezitose as diluent.

Forbes et al. describe in J. Aerosol Medicine (2000), 13(3), 281-288 the effects of pH, osmolarity, and lactose on epithelial permeability cell layers. Mannitol flux was used to assess epithelial permeability.

R. Boucher (The University of North Carolina) has filed a patent application (WO 00/36915) describing treatment of chronic obstructive diseases by administering an osmotically active compound such as a salt, sugar, sugar alcohol or organic osmolyte to the afflicted airway surface. The list of compounds is extensive—however only monosaccharides are among the carbohydrates mentioned per se in the claims i.e. osmolytically active and thereby teaches away from the present invention.

It has been established that an osmotic gradient across the respiratory epithelium results in morphologic changes in the epithelial cells and a widening of intercellular spaces. No significant changes in FEV₁ has been observed after inhalation of solutions with an osmolality between 159-549 mOsm (Am. Rev. Resp. Dis. (1982), 125 (suppl) 61).

The above references have highlighted the problem in selecting a pharmaceutical excipient by studying the effect of different pH, osmolarity and other parameters.

### DESCRIPTION OF THE INVENTION

In a first aspect the invention provides a pharmaceutical formulation in the form of an ordered mixture for respiratory administration comprising a drug and maltitol excipient.

Medicaments suitable for inclusion in the formulation of the present invention are any which may be delivered by inhalation.

The pharmacologically active agents in accordance with the present invention include glucocorticosteroids such as: budesonide, fluticasone (e.g. as propionate ester), mometasone (e.g. as furoate ester), beclomethasone (e.g. as 17-propionate or 17,21-dipropionate esters), ciclesonide, triamcinolone (e.g. as acetonide), flunisolide, zoticasone, flumoxonide, rofleponide, butixocort (e.g. as propionate ester), prednisolone, prednisone, tipredane, steroid esters according to WO 2002/12265, WO 2002/12266 and WO 2002/88167 (I) e.g. 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester and 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, steroid esters according to DE 4129535 (II) and the like. Long-acting β₂agonists, without limitation, include: salmeterol, formoterol, bambuterol, TA 2005 (chemically identified as 2(1H)-Quinolone, 8-hydroxy-5-[1-hydroxy-2-[[2-(4-methoxy-phenyl)-1-methylethyl]amino]ethyl]-monohydro-chloride, [R-(R*,R*)] also identified by Chemical Abstract Service Registry Number 137888-11-0 and disclosed in U.S. Pat. No. 4,579,854, formanilide derivatives (III) e.g. 3-(4-{[6-({(2R)-2-[3-(formylamino)-4-hydroxyphenyl]-2-hydroxyethyl}amino)hexyl]oxy}-butyl)benzenesulfonamide as disclosed in WO 2002/76933, benzenesulfonamide derivatives (IV) e.g. 3-(4-{[6-({(2R)-2-hydroxy-2-[4-hydroxy-3-(hydroxymethyl)phenyl]ethyl}amino)-hexyl]oxy}butyl)benzenesulfonamide as disclosed in WO 2002/88167 and the like. Several of these compounds could be administered in the form of pharmacologically acceptable esters, salts, solvates, such as hydrates, or solvates of such esters or salts, if any. Both raccemic mixtures as well as one or more optical isomers of the above compounds are within the scope of the invention.

The preferred pharmacologically active glucocorticosteroid agents for use in accordance with the present invention includes mometasone furoate, ciclesonide, zoticasone, flumoxonide, steroid (I), steroid (II) or, fluticasone propionate and budesonide, and even more preferred is budesonide. The preferred pharmacologically active long-acting β2-agonist is salmeterol xinafoate, formanilide derivatives (mB), benzenesulfonamide derivatives (I) and formoterol (e.g. as fumarate dihydrate) and even more preferred is formoterol fumarate dihydrate.

The preferred combinations include fluticasone propionate/salmeterol xinafoate, ciclesonide/formoterol fumarate dihydrate, mometasone furoate/formoterol fumarate dihydrate, fluticasone propionate/formoterol fumarate dihydrate, steroid (I)/formanilide derivative (III), steroid (I)/benzenesulfonamide derivative (V), steroid (D)/formoterol fumarate dihydrate, zoticasone/benzenesulfonamide derivative (MV) and zoticasone/formanilide derivative (M). The most preferred combination is budesonide/formoterol fumarate dihydrate.

Maltitol is widely used in the pharmaceutical industry in the formulation of oral dosage form. It has properties which make it suitable as an inhalation excipient. For example it is noncariogenic (i.e. not effecting your teeth) bulk sweetener, as sweet as sucrose, well adapted as a diluent for the different oral dosage forms, wet granulations and hard coating. It is obtained from hydrogenated maltose syrup (from starch). Maltitol also has good thermal and chemical stability. It does not undergo browning reaction with amino acids, and absorbs moisture only at relative humidities of 89% and above 20° C.

Maltitol is generally regarded as a nontoxic, nonallergenic and nonirritant material. A water solution is stable for at least 2 years at room temperature and pH 2-9! It is very stable at pH 4-9 even at higher temperatures. Maltitol is approved for food and non-parenteral pharmaceutical formulations in Europe and US.

The maltitol can be crystalline and in the form of anhydrate or different hydrates, if any. The crystalline maltitol is preferrably not spherical in shape.

Small particles of either drugs or excipients are often made by techniques such as micronization or grinding. Most methods create particles which are amorphous or having partially amorphous structures. These particles are liable to change their structure when kept in an adverse environment e.g. high humidity for a certain period of time. The end result is often a decrease in dispersibility and a reduced dose delivered to the patient One known process to resolving this problem is to reduce or eliminate the unstable amorphous phase by a conditioning process e.g. as described in EP 717 616 or U.S. Pat. No. 5,874,063. The same process could be used also for larger carrier/diluent particles.

The maltitol excipient may largely consist of much bigger particles (“coarse particles”) so that an “ordered mixture” may be formed between the active compound(s) and the excipient. The coarse particles may have a diameter of over 20 μm. Preferably, the coarse particles have a diameter of 60-800 μm. A further variation of such “ordered mixture” is a mixture of small particles below 10 μm of the excipient together with the coarser particles in combination with the active compound(s).

In the method for selection of new excipients we have included possible pharmacodynamic effects based upon the osmolytic behaviour of each compound—the reason being an effect on ciliary activity and on the reology of the mucus. Hyperosmolarity also triggers release of mediators from human mast cells e.g. histamine (Am. Rev. Resp. Dis, 137 (1988), 606). A clinical study involving fifteen stable asthma patients inhaling lactose dry powder alone or salbutamol added (no dosage data given) has also been reported (Eur. Resp. J. (1995), 8 (Suppl. 19, 426S)) where lactose caused bronchoconstriction, but the effect was masked since the rapid acting drug was added to the dry powder. If β2-agonists with slow onset or inhaled steroids are given with lactose dry powder as an excipient (carrier) substance this bronchoconstrictive effect could be a disadvantage, particularly with larger doses of excipient reaching the lung. However the effect is expected to be small.

When using a dry powder formulation a high local concentration of the components will be experienced. There is a risk to obtain a high local osmolarity causing bronchoconstriction or other adversed effects. The phenomena of osmolarity has not been a main issue in powder formulations for inhalation and particularly not in the selection of excipients for such formulations. These drawbacks have now been eliminated by the present invention, namely by selecting a chemical stable, non-hygroscopic excipient so as to minimize the risk for high local osmolarity and at the same time eliminate the risk for TSE thereby being suitable for inhalation.

Table 1 shows the concentrations of different excipients giving iso-osmotic solutions to saline i.e. the higher concentration the less possiblity for bronchoconstriction due to the excipient. We preferably select an excipient with a concentration of at least >5.5%, preferably >7%—compounds that could be regarded as weak osmolytic or non-osmolytic active compounds—teaching away from WO 00/36915. The selected concentration values are based upon the clinical results presented for lactose and mannitol. The physiological condition is pH 7.4 and 276 mOsm.

TABLE 1
Osmolarity for an aqueous solution (%) giving an
iso-osmotic solution with serum.¹

| | |
|---|---|
| Maltitol | 10 |
| Lactose | 9.8 |
| Sucrose | 9.3 |
| Lactitol | 7 |
| Ascorbic acid | 5.9 |
| Dextrose (glucose) | 5.5 |
| Sorbitol | 5.5 |
| Mannitol | 5.1 |
| Fructose | 5.1 |
| Oxymethazoline-HCl | 4.9 |
| Galactose | 4.9 |
| Xylitol | 4.6 |
| Lidocaine-HCl | 4.4 |
| Sodium ascorbate | 3.0 |
| Sodium chloride | 0.9 |

¹The Pharmaceutical Codex - Principles and Practice of Pharmaceutics, 12^{th} edition, London, 1994.

The osmotic pressure is proportional to the concentration of the solute for nonelectrolytes. The osmotic pressures of solutions of different nonelectrolytes are proportional to the number of molecules in each solution. This means—when having the same amount in grams—a disaccharide will have about half the osmotic pressure as a monosaccharide, which could also be seen in table 1. This type of generalisations could not be used for electrolytes. All disaccharides (e.g. trehalose) would be expected to have an osmotic pressure corresponding to a solution of at least >7% and trisaccharides (e.g. melezitose, raffinose) needed higher concentration in order to be iso-osmotic with a saline solution. The carbohydrate myo-inositol would be expected to have a concentration of less than 5% for iso-osmolytic activity with saline.

When the powder preparation of the present invention is intended for oral or nasal inhalation the formulation should consist of a) primary particles of active pharmacological drug particles having a diameter of less than 10 μm, for example between 0.01 to 6 μm or b) agglomerates of said particles.

The excipient in the formulation for oral or nasal inhalation may largely consist of particles having a diameter of less than about 10 μm so that the resultant powder as a whole consists of optionally agglomerated primary particles having a diameter of less than about 10 μm;

There are many factors that influence powder behaviour e.g. particle size and distribution, shape, crystallinity, charge density, chemical composition and environmental humidity. To cope with this, rigorous control of starting material and processes is required. Commonly used size reduction techniques including jet mill micronization, produce particles which may have regions of partially amorphous structure and which have an irregular shape. Such particles have a high surface energy and are liable to structural changes which may even include sintering if exposed to humidity during storage or use. The amorphous structure may be eliminated by subjecting the particles to a controlled conditioning process.

Loose particle agglomerates are formed as fine particles are exposed to movements within a powder bed. The ability of a powder to form agglomerates without additional binders is closely bound up with the adhesive forces. The agglomerates, as well as the ordered mixture, should be such as to give a sufficient adhesion force to hold the small drug particles during manufacturing, transportation etc but small enough to be broken during inhalation of the powder. A hygroscopic compound will strongly decrease this deagglomeration process and when the powder has been exposed to a high humidity. The result will be a low respirable dose delivered from the inhaler. The carrier particles should therefore be as less hygroscopic as possible and it is the object of this invention to link also this property to a selected excipient. The selected excipients according to the invention should be only slightly hygroscopic i.e. no moisture increase occurring below 80% relative humidity and the increase in moisture content, when the excipient is stored at 80% relative humidity or above for 1 week, does not exceed 40% according to the proposition by the Working Party “Guide for the technical content of monographs” of the European Pharmacopoeia Commission (Pharmeuropa, vol. 4, no 3 Sep. 1992, pages 228-230).

In order to eliminate chemical interactions as much as possible the excipient should be non-reducing e.g. not react when tested in Fehling's solution (Method of analysis, see Ph Eur 2001).

In a further aspect the invention provides a pharmaceutical formulation for respiratory administration as defined herein for use in a dry powder inhaler or a pressurised metered dose inhaler (pMDI).

1. A pharmaceutical formulation in the form of an ordered mixture for respiratory administration comprising a drug and maltitol excipient.

2. A formulation according to claim 1 where the excipient has not spherical shape.

3. A formulation according to claim 1, wherein the coarse particles may have a diameter of over 20 μm.

4. A formulation according to claim 1, wherein the coarse particles have a diameter of 60-800 μm.

5. A formulation according to claim 1, wherein the drug is selected from β2-adrenoreceptor agonists for example salbutamol, terbutaline, rimiterol, fenoterol, reproterol, adrenaline, pirbuterol, isoprenaline, orciprenaline, bitolterol, salmeterol, formoterol, clenbuterol, procaterol, broxaterol, picumeterol, TA-2005 and malbuterol and salts and hydrastes of such salts; anticholinergic bronchodilators for example ipratropium bromide, oxitropium and its salts and tiotropium and its salts; glucocorticosteroids for example beclomethasone, fluticasone, budesonide, tipredane, dexamethasone, betamethasone, fluocinolone, triamcinolone acetonide, flunisolide, mometasone and 16, 17-acetals of pregnane derivatives, for example rofleponide palmitate and ciclesonide and derivatives of these steroids; anti-allergic medicaments for example sodium cromoglycate and nedocromil sodium; leukotriene antagonists for example, zafirlukast, montelukast, pranlukast, and zileutoni antihistamines for example terfenadine, cetirizine, loratadine and azelastine; antibiotics; and pain control substances, for example morphine, codeine, pethidine.

6. A formulation according to claim 1 wherein the drug is selected from formoterol, terbutaline or budesonide and salts and hydrates thereof and hydrates of salts and a formoterol/budesonide combination e.g., Symbicort®.

7. A formulation according to claim 1, wherein a drug combination is selected from formoterol/budesonide; formoterol/fluticasone; formoterol/mometasone; salmeterol/fluticasone; formoterol/tiotropium salts; zafirlukast/formoterol; zafirlukast/budesonide; montelukast/formoterol; montelukast/budesonide; loratadine/montelukast and loratadine/zafirlukast and derivatives and salts and hydrates of such derivatives and salts.

8. A method of selecting a crystalline excipient having its origin from the vegetable kingdom or being totally synthesized for use as a carrier/diluent in the preparation of pharmaceutical formulations for respiratory administration of micronised drugs by means of an inhaler comprising
i) selecting an excipient that is a non-ionic compound, giving an iso-osmotic solution to saline when dissolved in water at a concentration of at least 5.5% (w/v) and
ii) being at the most only slightly non-hygroscopic and non-reducing.

9. A pharmaceutical formulation for respiratory administration comprising a drug and maltitol excipient.
